(19) United States
(12) Patent Application Publication
(10) Pub. No.: US 2010/0267105 A1
(43) Pub. Date: Oct. 21, 2010

(54) Method for Micromanipulation of Cells

(76) Inventors: Ellen Townes-Anderson, Morristown, NJ (US); Kormakur Hognason, East Orange, NJ (US)

Correspondence Address: LICATA & TYRRELL P.C., 66 EAST MAIN STREET, MARLTON, NJ, 08053, US

(21) Appl. No.: 12/761,740

(22) Filed: Apr. 16, 2010

Related U.S. Application Data
(60) Provisional application No. 61/212,979, filed on Apr. 16, 2009.

(51) Int. Cl. C12N 13/00

(52) U.S. Cl. 435/173.1

(57) ABSTRACT

The present invention is a method for manipulating living cells in vitro using poly-2-hydroxyethylmethacrylate-coated surfaces and optical tweezers to obtain cells in isolation or to create specific cell-cell interactions.

This application claims benefit of priority to U.S. Provisional Application Ser. No. 61/212,979, filed Apr. 16, 2009, the content of which is incorporated herein by reference in its entirety.

This invention was made with government support under Grant No. NEI 12031 awarded by the National Institutes of Health. The government has certain rights in the invention.

### INTRODUCTION

### Background of the Invention

Subsequent to neuronal determination, the differentiating nerve cell produces an axon that grows with relative accuracy to its designated postsynaptic cell. Target selection, which must occur before synaptogenesis, has been broken down into a number of steps, including defasiculation (for projection neurons), branching in the target region, finding the correct topographic location, terminating in the appropriate layer, and connecting with the appropriate cells within that layer (Holt & Harris (1998) Curr. Opin. Neurobiol. 8(1):98-105). These carefully orchestrated activities result in cell-specific patterns of connectivity.

In regeneration of the central nervous system, additional activities by growing processes must occur. Axons need to overcome mechanisms of inhibition, which are established after the brain and spinal cord are developed (Thanos & Thiel (1990) Graefes Arch. Clin. Exp. Opthalmol. 228(4):369-76; Baird et al. (1992) J. Neurobiol. 23(5):579-91). Functional recovery after CNS injury or disease includes target selection by the new processes. Just as regrowth of adult axons occurs when both a permissive environment and intrinsic mechanisms for growth are present, correct targeting presumably depends upon environmental and intrinsic factors. Not only must external cues be present, the cell must be able to respond to these cues. Transplantation of fetal mammalian cells into adult, injured brain, which results in selective axonal targeting by the transplanted cells, suggests that necessary environmental cues are present (Isacson & Deacon (1996) Neuroscience 75(3):827-37). However, even in the well-known retinotectal pathway of the goldfish, regenerating axons make many errors in their initial target area selection (Meyer & Kageyama (1999) J. Comp. Neurol. 409(2):299-312). In lizard, retinal ganglion cells grow to the tectum but are unable to find correct topographic locations to terminate (Beazley, et al. (1997) J. Comp. Neurol. 377(1):105-20). Thus, in repair of injury to the CNS, targeting may not proceed smoothly even when inhibition of growth has been overcome.

Many areas of the central nervous system are composed of multiple cell layers, such as the cortical and cerebellar layers, layers of the spinal dorsal horn and thalamic nuclei, and retinal layers. Progress is being made in understanding how neurons target their axons to specific layers in these structures during development (Sanes & Yamagata (1999) Curr. Opin. Neurobiol. 9(1):79-87). However, little is known about targeting to specific cells and therefore layer-specific recreation of connectivity after injury. Some of the lack of information in the adult CNS is due to the absence of model systems where targeting can be examined at the cellular level. A culture system of adult amphibian retinal cells that can be maintained in defined medium and in which functional synapses form have been used (MacLeish (1988) Neuron 1:751-760).

To examine the ability of adult sensory neurons, the cone and rod photoreceptors, to regenerate appropriate circuitry, analysis of single cell interaction is required to assess the targeting of photoreceptor axon outgrowth. The intact retina is composed of three cellular layers separated by two synaptic layers. Cone and rod photoreceptors synapse with second order horizontal and bipolar cells in the outer synaptic layer. The bipolar cells in turn interact with the third order (amacrine and ganglion) cells in the inner synaptic layer (Lasansky (1973) Philos. Trans. R Soc. Lond. B Biol. Sci. 265:471-489; Lasansky (1980) J. Physiol. 301:59-68; Wong-Riley (1974) J. Neurocytol. 3:1-33). This structure is consistent across all vertebrate species. Thus, correct targeting of adult photoreceptors would result in interactions with second order neurons, exclusively.

The issue of targeting in the adult retina takes on additional significance due to reports of neuritic sprouting by adult photoreceptors. This growth is observed in a variety of human retinal degenerations including retinitis pigmentosa (RP), age-related macular degeneration and retinal detachment (Townes-Anderson & Zhang (2006) Synaptic Plasticity and Structural Remodeling of Rod and Cone Cells. In: Pinaud et al., eds. Plasticity in the Visual System. New York: Springer, p 13-32; Marc, et al. (2006) From Genes to Circuits. In: Pinaud et al., eds. Plasticity in the Visual System. New York: Springer, p 33-54). In particular rod, but not cone, cells grow neurites with presynaptic varicosities filled with synaptic vesicles into the inner retina where the amacrine and ganglion cells are located. In an animal model of one form of RP, cone cell neuritic growth has been observed but much of this sprouting remains in the outer retina (Fei (2002) Mol. Vis. 8:306-14). The cause of this sprouting and the functional consequences to the diseased retina are completely unknown.

In a previous study, which examined randomly plated retinal neurons after two weeks in culture (Sherry, et al. (1996) J. Comp. Neurol. 376:476-488), it was discovered that a statistically significant preference for novel, third order, neurons as synaptic contacts of photoreceptor cells. This study suggested that in retina, at the level of cell recognition, correct targeting by photoreceptors did not occur. However, random platings of cells presented several technical problems. To insure the formation of cell pairs and groups, the cultures had been plated at relatively high density and multiple cells interacted with an individual photoreceptor. The cellular influences on these preferences, therefore, were probably multivariate making it difficult to know which cells or secreted cell products influenced targeting and contact formation. Additionally, it was not possible to identify all second and third order neurons. This led to significantly fewer groups with identifiable bipolar cells. Finally, cone and rod photoreceptors were not analyzed separately, in part because they were difficult to distinguish morphologically after two weeks in culture.

Such limitations can be overcome by the isolation of single cells with optical tweezers. Optical tweezers work by trapping a cell in a beam of infrared laser light. It has been shown that retinal cells can be manipulated by laser light without toxicity (Townes-Anderson, et al. (1998) Mol. Vis. 4:12). Redistribution of Chinese Hamster Ovary cells between poly-2-hydroxyethylmethacrylate-coated PDMS microwells has also been suggested using optically mediated particle clearing (OMPC) (Baumgartl, et al. (2009) Lab Chip 9:1334-1336).

### SUMMARY OF THE INVENTION

The present invention is a method for micromanipulating cells to place the cells in predetermined locations or a new environment, or in particular relationship with other cells, tissues or devices. The method involves the steps of placing a population of cells on a surface coated with poly-2-hydroxyethylmethacrylate; and micromanipulating the cells to a predetermined location on an adherent surface using optical trapping, wherein in some embodiments, the micromanipulation is performed to form a cell pair.

### BRIEF DESCRIPTION OF THE DRAWINGS

FIG. 1 shows the generation of cell pairs with optical tweezers. A rod cell on the poly-HEMA side of the culture dish (upper right) is selected, trapped in the laser beam and transported to the Sal-1 side where it is placed less than 10 μm away from a bipolar cell (upper left) to form a pair (lower middle). The bipolar cell is identified by the presence of a thick Landolt club (lc). The photoreceptor is identified by the presence of an ellipsoid.

### DETAILED DESCRIPTION OF THE INVENTION

Trapping forces of optical tweezers are generated from the momentum of light (Ashkin (1991) ASGSB Bull. 4(2):133-46; Ashkin, et al. (1986) Opt Lett. 11:288-290). Although these forces easily trap cells in suspension, they are not able to move cells that adhere to a surface. Experiments to manipulate retinal neurons with optical tweezers (Townes-Anderson, et al. (1998) supra) have employed a thin layer of SYLGARD (Dow Corning Co.) to reduce cell adhesion to the culture dish. However, there remains a need for improved techniques to reduce cell adhesion. It has now been demonstrated that poly-HEMA (poly-2-hydroxyethylmethacrylate), a nontoxic compound with cell repellent properties (Folman & Moscona (1978) Nature. 273:345-349), reduces cell adhesion to surfaces. Accordingly, the present invention features a method to micromanipulate, i.e., select, move, and position, living cells in vitro using poly-HEMA coated surfaces and optical tweezers to obtain cells in isolation or to create specific cell-cell interactions.

The method of this invention involves the steps of placing a population of cells (i.e., two or more cells) on a surface coated with poly-2-hydroxyethylmethacrylate; and using optically trapping to micromanipulate the cells to a predetermined location on an adherent surface. Cells which can be micromanipulated in accordance with the present method include, but are not limited to, neurons, lymphocytes, macrophages, microglia, cardiac cells, liver cells, smooth muscle cells, and skeletal muscle cells. In one embodiment, mammalian cells are used including, e.g., cultured cells such as Chinese Hamster Ovary Cells (CHO) cells, NIH-3T3, and HEK-293 cells and can express recombinant molecules (e.g., recombinant receptors and/or ion channels). However, bacterial cells (E. coli, Bacillus sp., Staphylococcus aureus, and the like), protist cells, yeast cells, plant cells, insect and other invertebrate cells, avian cells, amphibian cells, and oocytes, also can be manipulated. In some embodiments, the cells are eukaryotic. In other embodiments, the cells are somatic cells. In so far as the instant method does not involve direct contact with cells, this method finds particular application with cells containing numerous long protrusion and extensions, such as nerve cells with axons and dendrites. A population of cells can be prepared using conventional cell culture techniques, using cell culture lines, or from dissected tissues after, e.g., mechanical and/or enzymatic separation.

Cell manipulation requires overcoming cell adhesion to culture dish surfaces. Even contact with clean glass can prevent movement because of electrostatic interaction between the surface of the glass and the cell membrane. Conventional attempts to reduce attraction of the surface for cells last for only minutes. Use of poly-HEMA causes reduction of cell adhesion to culture surfaces for at least one hour. Accordingly, the population of cells of this invention is placed on a surface coated with poly-2-hydroxyethylmethacrylate (poly-HEMA). Such a surface can be a glass, polymeric, ceramic, metal or plastic surface or substrate (e.g., a coverslip or culture dish). As exemplified herein, the poly-HEMA can be applied to the surface as a thin, even coating suitable for repelling the adherence of the cells to the surface.

By way of illustration, poly-HEMA-coated surfaces can be prepared as follows. A coverglass is acid-cleaned coated with poly-HEMA (20 mg/ml 95% ethanol) by allowing a few drops of poly-HEMA solution to flow down the surface of the coverglass held at a steep incline thereby ensuring a thin, even coating. After drying in air in a dust-free environment, the coverglass is glued to the bottom of a 35 mm culture dish in which a hole approximately 1 cm in diameter has been drilled. A line indicating the edge of the poly-HEMA coating and 2 fiducial points are scratched on the bottom of the dish to act as reference points for locating cell position. The dishes are sterilized with UV light overnight.

Using optical trapping, a single cell can be obtained from a population of cells and moved to a predetermined location on an adherent surface. Optical tweezers (or laser tweezers), which rely on a highly focused single laser beam, are conventionally used to optically trap cells. Optical tweezers are known in the art for their use in manipulating biological materials, including cells. A basic optical tweezer typically includes the following components: a laser, a beam expander, some optics used to steer the beam location in the sample plane, a microscope objective and condenser to create the trap in the sample plane, a position detector (e.g., quadrant photodiode) to measure beam displacements and a microscope illumination source coupled to a CCD camera. See, e.g., Neuman & Block (2004) Rev. Scient. Instrum. 75:2787-2809; Dholakia, et al. (September 2002) Physics World μg 31-35. Optical tweezers can be obtained from a variety of commercial suppliers including, but not limited to, Aresis, Arryx, Elliot Scientific, JPK Instruments, MMI Optical Tweezers and Carl Zeiss.

An exemplary optical tweezer of use in the instant method has a 1 W, continuous wave diode laser of 980 nm wavelength mounted on a ZEISS AXIOVERT 100 inverted light microscope. Laser light is transmitted to the cells via an objective lens and focused at the same focal plane as the microscope. A high numerical aperture (N.A. 1.3) 40× oil immersion plan neofluor objective is used with brightfield optics for optical trapping. Computer software controls laser power and movement of the motorized stage, stores microscope stage coordinates, and creates macros for cell movement.

Using the optical tweezers, a single cell can be selectively moved with micron-level precision in a culture system to another location. With the instant method, cell selection and movement can proceed in a single culture for at least one hour, affording time to make multiple manipulations. According to this method, an isolated cell is moved to a predetermined location on an adherent surface. The surface can be made adherent to the isolated cell by coating the surface with sterile molecules known to be adhesive. Such molecules include, but are not limited to, laminin (adherent to neurons), poly-L-lysine, and ionene polymer (see U.S. Pat. No. 3,910,819).

While it is contemplated that the method of this invention can be adapted for use with a patch-clamp micropipette, subsequent release and culture of the cell is often difficult and can result in damage to the cell. Therefore, use of optical trapping in the instant method offers particular advantages for isolating and micromanipulating single cells.

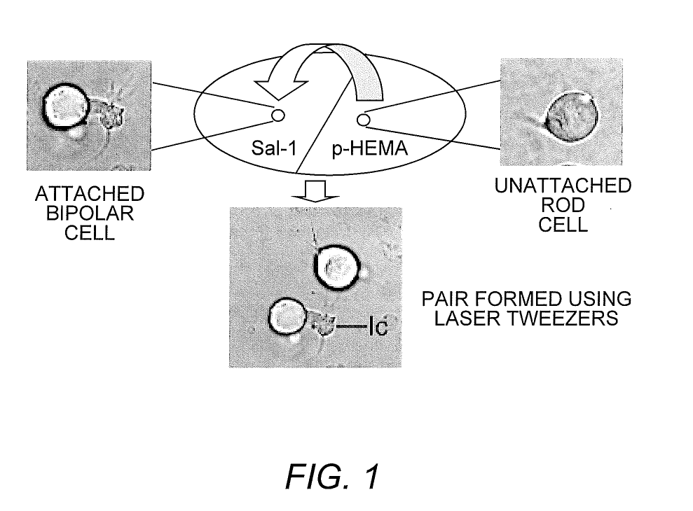

In addition to the isolation of single cells, the method of this invention can be used to create pairs of cells. For example, a photoreceptor cell type can be isolated and placed adjacent to a potential target cell, either a second order, bipolar, cell or a third order multipolar cell. Accordingly, in particular embodiments, the method embraces placement of the isolated cell of the invention adjacent to a second cell to form a cell pair. As an example of this embodiment, the following is a description of the formation of a nerve cell pair. First, a neuron is identified and selected on the adherent side of the dish (FIG. 1). The x and y stage coordinates of a position approximately 10 μm from the selected cell, is marked and saved to a computer. A neuron is then identified and selected on the nonadherent poly-HEMA side of the dish and optically trapped in the optical tweezers. Although trapping can be achieved over a broad range of power levels, micromanipulations can be routinely carried out using the laser at 10-20%, a setting low enough to avoid trapping debris but sufficient to transport the cell through the medium. While holding the nerve cell, the stage is lowered so that the cell is well above the surface of the culture dish and any attached neurons. The stage is then moved under computer control to bring the cell to the x,y co-ordinates set by computer. Stage movement is set at 8-20 μm/sec. Finally, the stage is raised to bring the trapped photoreceptor to the surface of the dish. The cell is placed within 2-10 μm of the selected cell's processes where it is allowed to adhere to the substrate. Digitized images of both cells in the pair before and after pair formation with the laser tweezers are obtained with a CCD camera mounted on the microscope. The newly formed cell pair can then be maintained in culture and cell interactions studied with a variety of techniques.

Micromanipulation by optical trapping allows the maintenance of sterility. Because the laser beam goes through transparent surfaces of the culture dish, cell selection and placement can be done in an enclosed, sterile environment. Cell populations can be of low density because pairings depended on micromanipulation, not chance association. Moreover, distances between cells can be standardized because of the micron-level control of tweezers micromanipulation. Therefore, the present method finds application in the precision placement of cells on electrodes on a microelectrode array (MEA), the creation of cell-electronic prosthetic devices for biomedical purposes, precision creation of heterogeneous cultures, which may be useful to create tissues for transplantation, and precision creation of neural circuits or specific cell-cell groups for study of cell or group properties and cell trapping for precision transplantation in transparent tissue like neural retina.

The following non-limiting examples are provided to further illustrate the present invention.

### Example 1

### Materials and Methods

Preparation of Culture Dishes for Optical Tweezers. Trapping forces of optical tweezers are generated from the momentum of light (Ashkin (1991) supra; Ashkin, et al. (1986) supra). Although these forces easily trap cells in suspension, they are not able to move cells that adhere to a surface. In initial experiments to manipulate retinal neurons with optical tweezers (Townes-Anderson, et al. (1998) supra), a thin layer of SYLGARD was used to reduce cell adhesion to the culture dish. For the current studies, poly-HEMA (poly-2-hydroxyethylmethacrylate), a nontoxic compound with cell repellent properties (Folkman & Moscona (1978) supra), was used.

Acid-cleaned, #1 glass coverglass (VWR Scientific Inc., Media, Pa.) was prepared so that one half was coated first with poly-HEMA (20 mg/ml 95% ethanol, Sigma Chemical Co. St Louis Mo.). Allowing a few drops of poly-HEMA solution to flow down the surface of the coverglass held at a steep incline ensured a thin, even coating. After drying in air in a dust-free environment, the coverglass was glued with SYLGARD 184 (Dow Corning Co., Midland, Mich.) to the bottom of a 35 mm culture dish in which a hole approximately 1 cm in diameter had been drilled. A line indicating the edge of the poly-HEMA coating and 2 fiducial points were scratched on the bottom of the dish to act as reference points for locating cell position. The dishes were sterilized with UV light overnight. The other half of the coverglass was made adhesive to salamander retinal neurons by coating with sterile goat anti-mouse IgG antibody (0.1 mg/ml, Boehringer Mannheim Corporation, Indianapolis, Ind.) and then, after rinsing with sterile Ringer's, Sal-1 supernatant containing mouse anti-salamander antibody raised against retinal cell membranes (MacLeish, et al. (1983) Proc. Natl. Acad. Sci. USA 80:7014-7018). Dishes were incubated at 10° C. overnight and then rinsed with Ringer's to remove Sal-1 prior to introduction of 2 ml of serum-free salamander medium containing: 108 mM NaCl, 2.5 mM KCl, 2 mM HEPES, 1 mM NaHCO₃, 0.5 mM NaH₂PO₄, 1 mM sodium pyruvate, 0.5 mM MgCl₂, 24 mM glucose, 1.8 mM CaCl₂, 7% medium 199, 1 mM minimum essential (MEM) vitamin mix, 0.1 mM MEM essential amino acids, 0.1 mM MEM nonessential amino acids, 2 mM glutamine, 2 μg/ml bovine insulin, 1 μg/ml transferrin, 5 mM taurine, 0.8 μg/ml thyroxine, 10 μg/ml gentamycin, and 1.0 mg/ml bovine serum albumin (MacLeish (1988) supra; Mandell, et al. (1993) J. Neurosci. 13:3533-3548).

Preparation of Cell Cultures. Retinal cell cultures were obtained from light-adapted, adult, aquatic-phase tiger salamanders (Ambystoma tigrinum) 17-22 cm in length (Charles Sullivan Inc., Nashville, Tenn.). The animals were maintained at 5° C. on a 12-hour light/12-hour dark cycle for at least 1 week before experimentation. Cultures of retinal cells were prepared according to procedures in the art (Nachman-Clewner & Townes-Anderson, et al. (1996) Neurocytol. 25(10):597-613). Briefly, the animals were decapitated and pithed, and the retinas removed in room light. The retinas were subjected to enzymatic digestion with papain (14 U/ml, Worthington, Freehold, N.J.) in salamander Ringer's solution (85 mM NaCl, 1.5 mM KCl, 25 mM NaHCO₃, 0.5 mM CaCl₂, 0.5 mM NaH₂PO₄, 24 mM glucose, 0.03 mM phenol red, 1.0 mM sodium pyruvate) containing 2.7 mM DL-cysteine for 45 minutes at room temperature (20-22° C.) After rinsing, retinas were gently triturated with a 3 mm diameter wide-bore pipette to yield a cell suspension containing a mixture of photoreceptors, second-, and third-order neurons as well as Müller cells.

Laser Tweezers Manipulation of Retinal Cells. The optical tweezers-microtool or laser tweezers used for manipulation (Cell Robotics Inc., Albuquerque, N. Mex.) composed of a 1 W, continuous wave diode laser of 980 nm wavelength mounted on a Zeiss Axiovert 100 inverted light microscope. Laser light was transmitted to the cells via an objective lens and thus was focused at the same focal plane as the microscope. A high numerical aperture (N.A. 1.3) 40× oil immersion plan neofluor objective (Zeiss) was used with brightfield optics for optical trapping. Computer software (Cell Robotics Inc.) controlled laser power and movement of the motorized stage, stored microscope stage coordinates, and created macros for cell movement. A culture dish was placed on the microscope stage; cells were plated onto both halves of the prepared coverglass and allowed to settle for approximately 20 minutes, which was long enough for cells on the adherent side of the dish to attach to the antibody substrate.

Freshly isolated cells were identified predominately by cell shape (Sherry, et al. (1996) supra). Third order neurons, the multipolar amacrine and ganglion cells, were identified by the presence of one or more processes emanating from the cell body. Amacrine and ganglion cells were not distinguished from each other in this study; these neurons were classed as multipolar cells. Bipolar cells in salamander retina have a large primary dendrite known as a Landolt club from which secondary dendrites emerge. This feature is characteristic of all bipolar neurons. Horizontal cells, although identifiable, were not used in this study because of their low abundance. Photoreceptors can be identified by the presence of the ellipsoid, an accumulation of mitochondria, in the inner segment. Cone and rod cells can be distinguished from each other by overall shape, hourglass versus rounded respectively, and for the rod cells, when retained, the presence of an axonal fiber with synaptic pedicle(s). For cone cells, there is no axon fiber and cone cells therefore are assumed to have their presynaptic terminal, which lies at the nuclear pole.

To create cell pairs, an isolated second-order bipolar, or third-order amacrine or ganglion cell was selected on the Sal-1 adherent side of the dish (FIG. 1). The x and y stage coordinates of a position approximately 10 μm from the primary dendrites or Landolt club of the selected cell, was marked and saved to computer disk. A photoreceptor was then found on the nonadherent poly-HEMA side of the dish and optically trapped in the laser tweezers. Only photoreceptors without outer segments were selected for study. Although trapping could be achieved over a broad range of power levels, for the micromanipulations herein, the laser was used at 10-20%, a setting low enough to avoid trapping debris but sufficient to transport the cell through the medium. While holding the photoreceptor cell, the stage was lowered so that the cell was well above the surface of the culture dish and any attached neurons. The stage was then moved under computer control to bring the cell to the x,y co-ordinates set by computer as described above. Stage movement was set at 8-20 μm/sec. Finally, the stage was raised to bring the trapped photoreceptor to the surface of the dish. The cell was placed within 2-10 μm of the selected cell's processes where it was allowed to adhere to the Sal-1 substrate. Digitized images of both cells in the pair before and after pair formation with the laser tweezers were obtained with a CCD camera mounted on the microscope. The cell pairs were maintained in a humidified chamber at 10° C. in the dark for 7 days. Each newly formed pair was monitored for growth on a daily basis. On the seventh day cultures were fixed for immunocytochemistry for further identification.

Immunocytochemistry. Rod and cone cell identification was confirmed by the presence and absence, respectively, of rod opsin immunostaining using the monoclonal antibody 4D2 (Hicks & Molday (1986) Exp Eye Res. 42:55-71). This antibody recognizes the opsin in red (M) rod cells (Sherry, et al. (1998) Vis. Neurosci. 15(6):1175-87). A mouse monoclonal antibody against Goα (MAB #3073, Chemicon International, Temecula, Calif.) was used to stain ON-bipolar cells (Vardi (1998) J. Comp. Neurol. 395(1):43-52). Both antibodies have been previously characterized in salamander retina (Mandell (1993) supra; Zhang & Wu (2003) J. Comp. Neurol. 461:276-289). Cells were fixed with 4% paraformaldehyde in 0.125 M phosphate buffer, pH 7.4, for hours at 4° C. Procedures for immunocytochemistry have been previously reported (Mandell (1993) supra). Briefly, cells were washed with PBS (450 mM NaCl, 20 mM sodium phosphate buffer, pH 7.4), then incubated in goat serum dilution buffer (GSDB; 16% normal goat serum, 450 mM NaCl, 0.1% TRITON X-100, 20 mM phosphate buffer, pH 7.4) to block nonspecific binding and permeabilize the plasma membrane. The cells were then incubated with the primary antibody (4D2 1:25, Goα 1:1000 dissolved in GSDB) and maintained at 4° C. overnight. For negative controls, no primary antibodies were added to the GSDB at this step. Cells were rinsed with wash buffer (450 mM NaCl, 0.3% TRITON X-100, and 20 mM phosphate buffer) followed by a rinse with PBS. Then the cells were incubated with TRITON-free GSDB for 1 hour at room temperature followed by incubation with ALEXA 594-conjugated goat anti-mouse IgG (1:35, Molecular Probes, Eugene, Oreg.) secondary antibodies in TRITON-free GSDB for 60 minutes at room temperature in the dark. Cells were washed with PBS followed by a final rinse with 5 mM phosphate buffer, pH 7.4, and mounted in anti-fade medium composed of 90% glycerol, 10% PBS, and 2.5% (w/v) 1,4-diazobicyclo[2,2,2]octane.

Analysis of Photoreceptor Cell Growth. Analysis was performed on the digitized images of cone and rod cells taken using conventional phase-contrast and fluorescence microscopy, respectively. Because of the larger numbers and finer processes of rods, it proved easier to carry out measurements on the immunostained cells. Any outgrowth extending >5 μm from the soma or from lamellipodial-like processes extending from the soma was considered to be a primary process. Formation of a varicosity along a primary process demonstrated differentiation into a neuritic process. These varicosities are known to be filled with synaptic vesicles capable of recycling in response to depolarization (Mandell & Banker (1996) J. Neurosci. 16(18):5727-40). For quantification, a varicosity was defined as a swelling along a neuritic process with a diameter >1 μm (Mandell (1993) supra). Distance between cells after plating and again after 7 days in vitro was examined along the axis connecting the nuclei. Measurements were made double blind using Image-Pro Plus v4.1 (Media Cybernetics, Silver Spring, Md.).

To determine attraction and repulsion, a straight line was drawn connecting the centers of both cells in a pair. Cell center was determined by averaging the x and y axes. Cones were then divided into halves by drawing a single line through the cell's center that intersected the first line at right angles. Because rod cells have many more processes (Nachman-Clewner, et al. (1999) J. Comp. Neurol. 415(1):1-16), they were divided into equal sized quadrants by superimposing a cross consisting of two lines each subtending 45° with respect to the line joining the cells' centers and intersecting at the photoreceptor cell center. Measurements of cell growth were made within the half (cones) or quadrant (rods) facing toward the target cell and away from the target cell and then compared. Whether a photoreceptor was attracted or repelled by the target cell was determined by i) examining for cell contacts, ii) calculating the difference between the number of presynaptic varicosities formed by the photoreceptor in the direction towards and away from the target neuron, iii) calculating the difference between the numbers of primary neurites and the total neurite length toward or away from the target, and iv) examining for reduction or expansion of the distance between the photoreceptor and paired second- or third-order cell bodies. The formation of contact and the location of varicosities were of primary importance in determining attraction; thus, growth that resulted in broad contact between the photoreceptor and target cell somas was considered attraction. If there was no contact or no development of varicosities then process numbers and length toward and away from the target cell and changes in the distance between cells were the parameters used. Contacting processes were not included in this measure to avoid redundancy and because their growth potential was naturally limited by the contact. A minimum of two of the four types of measurements had to demonstrate differences in order to consider that attraction or inhibition had occurred. If quantification of growth did not reveal clear attraction or inhibition, the photoreceptor response to the target neuron was considered undetermined.

Analysis of Photoreceptor Orientation. After placement, micromanipulated photoreceptors were at different orientations with respect to the target cell. Cell polarity for photoreceptors was determined by the position of the nucleus and the ellipsoid, the accumulation of mitochondria in the inner segment. Cell orientations were subdivided into three groups: 0 degrees (photoreceptor facing the target with its nuclear pole; the nuclear pole comprised the surface of the cell within 45 degrees either side of the center of the nucleus), 180 degrees (photoreceptor facing the target with its ellipsoid pole, defined as the surface of the cell 45 degrees either side of the center of the ellipsoid), and 90 degrees (cell is sideways). Changes in cell polarity were determined for each cell pair by comparing the orientation at day 1 with that at day 7.

Statistical Analysis. Graphs were created using SIGMAPLOT v.5.0 (SYSTAT Software Inc., Chicago, Ill.). For percentage comparisons among groups, exact binomial or chi square analysis was employed using SAS version 8.0 (SAS Institute Inc., Cary, N.C.) software. Statistical comparisons of continuous measures between two groups used the Student's t-test if normality and equal variance tests were not rejected. Otherwise, the nonparametric Mann-Whitney rank sum test was used with SIGMASTAT v.2.0 (SYSTAT Software Inc., Chicago Ill.). The data were expressed as mean±SEM.

### Example 2

### Interaction Between Photoreceptors and Neurons

To examine the effect of cell type on photoreceptor targeting, pairs of rod-bipolar (second-order), rod-multipolar (third-order), cone-bipolar, and cone-multipolar cells were created. Optical tweezing was done within hours after retinal dissociation and cell plating and thus before any modification in cell shape. Although the neurons sustain some loss of cell processes during isolation, cell types remain distinct. Only cells positively identified were used to create cell pairs. Cell identification was augmented by immunocytochemistry: anti-rod opsin immunolabel distinguished rod from cone cells; anti-Goα antibody labeled most ON bipolar cells.

In culture, photoreceptors initially create actin-filled filopodia which emanate from all points on the cell's circumference (Mandell (1993) supra). Lamellipodia appear as well, frequently formed from existing synaptic pedicles (Nachman-Clewner & Townes-Anderson (1996) supra). Actin- and tubulin-filled neuritic processes develop from filopodia or regions of lamellipodia. Neurites subsequently develop synaptic-filled varicosities at their tips or along their length; varicosity-bearing processes can grow from any point along the cell soma (Sherry, et al. (1996) supra; Mandell (1993) supra). Although the processes extend and retract, their growth is not controlled by typical growth cones, as would appear on projection neurons. The neurites extend a maximum of approximately 50 μm, 2-3 times cell soma diameter. Thus placing the photoreceptors within 2-10 μm of a target cell insured that the targets were well within their growth range. Cell pairs were followed for a week, a period of vigorous photoreceptor process growth but before functional synaptic development (MacLeish (1988) supra). During this time, photoreceptors grew neuritic processes, developed presynaptic varicosities, and in some cases formed cell contacts, consistent with previous studies (Sherry, et al. (1996) supra). Control pairs, created without micromanipulation and composed of photoreceptors next to a second or a third order neuron, were identified in the same cultures used for tweezers micromanipulation.

Attraction or inhibition between cells can be initiated and/or maintained by secreted factors. To reduce the effects of extraneous soluble factors, cultures were plated at low density (range 16.3-343 cells/mm², mean 90.6 cells/mm²) so that pairs were at least one hundred microns away from other neurons. Any pair in which a third neuron came in contact with one of the members was discarded. However, no attempt was made to limit diffusion from the target cells over the 7-day period and thereby enhance possible gradients of guidance factors (Lumsden & Davies (1983) Nature 306(5945):786-8; Brown, et al. (2001) The developing brain Oxford, New York: Oxford University Press). Thus, with a prolonged period for diffusion of potential guidance factors, an all-or-nothing effect on the direction of photoreceptor growth was not anticipated.

The photoreceptors were analyzed by examining for cell contacts, and by quantifying changes in the number of presynaptic varicosities which formed toward or away from the partner, the number and total length of processes which grew toward or away from the potential postsynaptic partner, and the distance between cells over time. Although adult retinal neurons do not migrate in culture, in some cases of attraction, cells appeared to move together due to asymmetric expansion of the cell soma so that there was broad contact between cell bodies. Based on these measures, a photoreceptor cell was classified as attracted to or repulsed by its partner or undetermined. The undetermined category contained 1) photoreceptor cells that had neutral growth, i.e., equal amount of growth in all directions, 2) cells that had equal growth both towards and away from the target, i.e., cells that may have sensed multiple attractive or inhibitory molecules or may have been paired with a target cell which released weak or mixed signals, and 3) cells which responded poorly, i.e., cells which had little growth making it difficult to assess growth patterns. It is known that about 1-5% of the photoreceptors fail to grow well in culture. Two hundred and three pairs were analyzed from 86 cultures derived from 55 animals.

Observation of the pairs throughout the 7 days in vitro showed that retraction of neurites after process outgrowth was rare, indicating that intercellular effects were relatively stable over time. Cone cells grew an average of 6.4±0.3 processes per cell and formed on average 1.4±0.1 presynaptic varicosities. From analysis of growth patterns, cone cells were found to be attracted to and repulsed by both bipolar and multipolar cells but in distinctly different proportions. In cone-bipolar cell pairs (n=55), 53% showed attraction and 27% showed repulsion with the remaining pairs classed as undetermined; cones, therefore, showed an overall attraction to appropriate second order targets. For pairs with third order cells (n=43), 38% showed attraction and 56% showed repulsion, with 11% undetermined, indicating a repulsion of cone cells to inappropriate, multipolar targets. Rod cells grew an average of 35.3±1.5 processes and formed 5.8±0.6 varicosities per cell. For rod cells, 52% were attracted and 28% were repulsed by their bipolar partner (n=74); like cone cells, rod cells showed an attraction to normal second order targets. However, 65% of rod cells were attracted but only 10% were repulsed by multipolar partners (n=40), indicating an additional attraction to novel, third order cells. Of the pairs between rod and multipolar neurons showing attraction, 25% had formed broad cell-to-cell contact compared to 13% of attracted cone-multipolar cell pairs. This broad cell/cell contact was not due to fortuitous close pairing of cells: there was no statistical difference at day of plating in the intercellular distance between cells which subsequently showed attraction compared to those that subsequently showed repulsion. Moreover, pairs with broad contact were observed equally often in rod-bipolar and cone-bipolar cells (28 and 29% of attracted cell pairs respectively), indicating that cone cells had the ability to make broad cell contacts. Attraction to multipolar cells by rod cells appeared to be strong both on the basis of the number of pairs showing attraction and the number of attracted pairs showing broad cell-cell contact. With chi-square analysis, the attraction of rod cells to multipolar cells was significantly greater than to bipolar cells (p=0.027). Thus, there was an obvious difference in the effect of multipolar cells on growth and varicosity formation of cone and rod cells. This comparison between photoreceptor cell types is particularly striking when looking at the net effects of partners (% attraction minus % repulsion).

### Example 3

### Interactions Between Photoreceptors and Bipolar Cell Subtypes

It was contemplated that although layer-specific markers associated with specific cell classes may determine targeting, cell subtypes may also influence the outcome of cell class pairings. To investigate the effects of cell subtype, photoreceptor-bipolar interactions were examined because these second order neurons are divided into two basic categories, the ON and OFF cells. This division depends on the response to light: ON cells are active in the light; OFF cells are active in the dark. The functional differences are due in part to the differential presence of metabotropic and tonotropic glutamate receptors on ON and OFF cells respectively. In adult tiger salamander, ON and OFF cells are approximately equal in number (Maple, et al. (2005) Vision Res. 45:697-705). Since, for both cone and rod cells, about half the bipolar cells were attractive targets, it is possible that either the ON or the OFF cells were the preferred target cell subtype within the bipolar cell class.

The same cultures as above were re-examined for bipolar subtype interaction by immunolabeling. In salamander retina, ON bipolar cells have either predominantly cone input, more equally mixed rod and cone input or predominantly rod input (Wu, et al. (2000) J. Neurosci. 20:4462-4470). The presence of a unique receptor-associated G protein, Go, allowed us to positively distinguish most ON subtype cells. Staining for the alpha subunit of Go protein (Goa) is present in cone-dominated bipolars and mixed rod-cone bipolars, together comprising about 41% of all bipolar cells (Zhang & Wu (2003) supra). The other 59% of bipolar cells, which are not immunoreactive for Goα, are composed of the rod-dominated ON bipolars and all OFF cells. After fixation, cultures, which had been immunostained for rod opsin to distinguish rod from cone cells, were restained for Goα to distinguish ON and OFF bipolar cells. There were 113 photoreceptor-bipolar pairs. For each category, attraction, repulsion, and undetermined, the number of Goα-positive and -negative cells were counted. Both Goα-positive and -negative bipolar cells were present in each category; however, Goα-positive cells were more attractive than repulsive. For cones, 58% of Goα-positive cells were attractive, 29% were undetermined, and 13% were repulsive. For rod cells, 58% of Goα-positive cells were attractive, 23% were undetermined, and 19% were repulsive. Chi square analysis confirmed that cone and rod cells were more attracted than repulsed by Goα-positive cells (p<0.03 and p<0.02, respectively). In contrast, Goα-negative cells were approximately equally attractive and repulsive for both cone and rod cells (for cone cells, 44% versus 39%; for rod cells, 45% versus 35%). The data indicate a dependence upon bipolar cell subtype in neurite targeting, with the ON bipolar subtype providing a significantly attractive target for growth arising from both cone and rod cells. Thus, cell subtypes were not equally involved in targeting.

The number of Goα-positive cells present in the cone-bipolar cell pairs was greater than in the rod-bipolar cell pairs. To ensure that the numbers of Goα-positive versus Goα-negative bipolar cells used as target cells did not skew the results, the pool of bipolars presented to cone and rod cells were examined. Based on staining in the intact retina, the ratio of Goα-positive:negative cells should be approximately 41:59%. Unexpectedly, the pool of bipolar cells paired with cone cells contained 31 Goα-positive and 18 Goα-negative cells indicating that the Goα-positive cells were significantly more than 41% of the total pool (p<0.05). This is in contrast to the pool of cells paired with rod cells. For pairs between rod and bipolar cells there were 24 Goα-positive and 40 Goα-negative cells, very close to the 41:59% of Goα-positive:negative cells present in the intact retina. It is possible that cone cells caused Goα-negative cells to die; however, there were not adequate numbers of dying bipolar cells in all the created cone-bipolar cell pairs to account for the disproportionately large number of Goα-positive cells. Alternatively, cone cells may have stimulated or maintained an upregulation of Goα in bipolar cells.

For rod cells, even though less than 40% of the bipolar cell targets were Goα-positive, bipolar cells expressing Goα were more attractive than repulsive. The attraction of rod cells to Goα-positive cells is surprising when one considers the connectivity in the outer plexiform layer of the salamander retina. In salamander retina, all cone cells but only about 30% of rod cells (range 25-35%) contact Goα-containing cells (Zhang & Wu (2003) supra). This circuitry would suggest that Goα-containing bipolars would have limited attraction for rod cells. Instead, rod cells in vitro were attracted to versus repulsed by Goα-containing cells by almost 5:1 (14 attracted: 3 repulsed Goα-positive cells). Thus, significantly more rod cells than expected (p=0.001) were attracted to Goα-containing cells. Further, it indicates that some rods were contacting novel cell subtypes.

### Example 4

### Effects of Cell Target on Photoreceptor Growth

The preferences demonstrated in culture may be influenced by general stimulation or inhibition of photoreceptor growth by potential postsynaptic partners. This would result in greater growth from photoreceptor cells attracted to their partner and possibly less growth with inhibition, which might make it more difficult to detect repulsion. Therefore, the number of varicosities and the number and total length of processes produced by attracted versus repulsed photoreceptors were compared. Surprisingly, repulsive cells did not reduce total photoreceptor growth. Instead, the average number of varicosities, neurites and total length of neuritic growth did not differ regardless of whether the cells were attracted to or repulsed by a partner cell. Additionally, qualitative assessment of the amount and direction of growth by target cells suggested that growth by target cells did not determine photoreceptor targeting: abundant growth by target cells toward the photoreceptor could still result in repulsion of photoreceptor growth whereas no growth by a target cell could result in attraction.

Although there were no statistical differences in the total amount of growth in attracted and repulsed pairs, there was a trend toward more neuritic development in attracted pairs. Previous work had shown an increase in the number of varicosities after cell contact in 2 week-old cultures (Sherry, et al. (1996) supra). Thus, the effect of contact on photoreceptor growth parameters was examined. In the 1-week cultures, a significant increase in varicosities was present in photoreceptors that contacted target cells (p<0.05). About two thirds of all photoreceptors. which made contacts. produced varicosities. When examined separately, there was a significant increase in varicosities per cone cell, by 91%, with cell contact and per rod cell by 54%, with contact, if only rod cells that made varicosities were examined. The data indicate that regeneration of synaptic interaction occurs in two steps: 1) potential partners secrete a factor guiding neuritic growth but not determining total amount of growth and 2) if contact is established axonal differentiation in the form of presynaptic development is stimulated.

### Example 5

### Effects of Cell Target on Photoreceptor Polarity

Technical issues related to optical tweezing may also affect partner preferences. When cells are placed next to each other, the nuclear side of the photoreceptor from which the axon normally emerges does not always face the dendritic pole of the second or third order neuron. Because photoreceptors can grow processes from any point of the cell body (Mandell (1993) supra), orientation was not expected to influence the direction of cell growth. However, to test for the effects of orientation, rod and cone cells were analyzed for polarity (location of the ellipsoid, an accumulation of mitochondria, with respect to the nucleus determines the axis of the cell). Where the nuclear pole faced, toward or away from the target cell, was assessed at day 1 and day 7. No effect of initial polarity on final attraction or repulsion was seen. For example, less than a third of cones paired with a bipolar partner was correctly oriented one day after plating whereas more than 40% of cone cells paired with multipolar cells was correctly oriented. If polarity determined preference then cone cells should have preferred multipolar over bipolar cells; the opposite was in fact observed. By day 7, however, the polarity of some cell pairs unexpectedly had changed. Changes were usually gradual, occurring over several days in culture. For photoreceptors whose polarity changed, there was a significant association between attraction and repulsion and change towards or away from the target respectively (p=0.01).

Finally, pairs that were formed randomly in the culture dish, without the use of optical tweezers, were examined as internal controls for cell targeting (n=23). As previously reported (Townes-Anderson, et al. (1998) supra), the use of optical tweezers did not change the morphology or amount of process outgrowth. The proportion of attracted and repulsed cell pairs based on cell type was not significantly different than for tweezers-manipulated pairs (p>0.05). The majority of rod cells, for instance, were attracted to multipolar cells.

Optical tweezers were used herein to pair identified cells under sterile culture conditions and test for regenerative interactions. In contrast to previous studies using randomly plated cells (Sherry, et al. (1996) supra), the present method allowed for analysis of rod and cone cells separately and creation of adequate numbers of pairs with bipolar and multipolar cells. Examination of pairs that grew for 7 days demonstrated that rod and cone cells have different target preferences. Cone cells preferred to grow toward their normal partners, bipolar cells, when forming new neuritic sprouts; among bipolar cells, they were more attracted to ON than to OFF cells. In contrast, rod cells sought novel interactions. Their preferred partner was a third order neuron, a novel target. Within the bipolar class, rod cells sought out Goα-positive-bipolars, a bipolar subtype that normally interacts with only a fraction of the rod cells in vivo and, therefore, would also be a novel target to most rod cells. Thus, cell types considered to be closely related morphologically and functionally, demonstrate very different abilities to target appropriately in culture.

This study also demonstrated the feasibility of using optical tweezers to examine neuronal growth. In the cultures herein, non-manipulated cell pairs had similar target preferences to pairs made by micromanipulation. Further, initial cell orientation, after placement of a cell by the tweezers, did not determine target preference. Thus, tweezers manipulation itself did not appear to influence cell/cell interactions. These techniques, therefore, are applicable to any type of neuron and allow formation of groups of various sizes and composition.

What is claimed is:

1. A method for micromanipulating cells comprising
(a) placing a population of cells on a surface coated with poly-2-hydroxyethylmethacrylate; and
(b) micromanipulating the cells to a predetermined location on an adherent surface using optical trapping thereby micromanipulating the cells.

2. The method of claim 1, wherein the micromanipulation is performed to form a cell pair.
